(19) United States
(12) Patent Application Publication
(10) Pub. No.: US 2004/0142328 A1
(43) Pub. Date: Jul. 22, 2004

(54) Method and cloning vector for preparing multiple-gene diagnostic probes for the assessment of multiple markers for breast cancer prognosis

(75) Inventors: Richard Idem Somiari, Johnstown, PA (US); Stella Bassey Somiari, Johnstown, PA (US); F. Nicholas Jacobs, Davidsville, PA (US); Rick Jordan, Johnstown, PA (US)

Correspondence Address: STEPTOE & JOHNSON LLP, 1330 Connecticut Ave., NW, Washington, DC, 20036, US

(73) Assignee: WINDBER RESEARCH INSTITUTE, Windber, PA

(21) Appl. No.: 10/337,231

(22) Filed: Jan. 6, 2003

(51) Int. Cl. C12Q 1/68; C12P 19/34

(52) U.S. Cl. 435/6; 435/91.1

(57) ABSTRACT

The problem of inadequate and cost ineffective broad-range prognostic factors for breast cancer is solved by providing a method for constructing a cloning vector for preparing a multi-gene probe for a single-step determination of disease outcome. The 5-genes chosen (HER2, Topo IIα, NM23-H1, CK19 and MMP9) are known to show altered expression in different breast tumors. The multi-gene probe is labeled and used to screen a tumor specimen. Since five prognostic markers are prepared and used simultaneously, a wider variety of breast cancers may be covered and disease outcome prediction may be improved in a wider population at a substantially reduced cost.

### CROSS-REFERENCE TO RELATED APPLICATION

This application is related to Ser. No. 10/237,614, filed Sep. 10, 2002, for “Multiple-Gene Diagnostic Probes And Assay Kits And Method For The Assessment Of Multiple Markers For Breast Cancer Prognosis” by the present inventors, the disclosure of which is expressly incorporated herein by reference.

### STATEMENT REGARDING FEDERALLY SPONSORED RESEARCH OR DEVELOPMENT

This invention was made with government support under the Clinical Breast Care Project, Contract Number 17330, awarded by the Henry M. Jackson Foundation For the Advancement Of Military Medicine as a subcontract under Grant Number MDA 905-00-1-0022 from the Uniformed Services University of the Health Sciences.

### BACKGROUND OF THE INVENTION

1. Field of the Invention

The present invention relates to a method for preparing multiple-gene diagnostic probes for the assessment of multiple markers for breast cancer prognosis. In addition, the invention relates to a cloning vector for preparing multi-gene probes for the assessment of multiple markers for breast cancer prognosis.

2. Description of the Related Art

Cancer is a disease that results when the controls that regulate normal cell growth break down. The growth and development of normal cells are subject to a multitude of different types of control. A fully malignant cancer cell appears to have lost most, if not all, of these controls. However, conditions that seem to represent intermediate stages, when only some of the controls have been disrupted, can be detected. Thus, the progression from a normal cell to a malignant cell is a multistep process, each step corresponding to the breakdown of a normal cellular control mechanism.

Normal growth controls appear to become ineffective because of mutations in the cellular genes coding for components of the regulatory mechanism. Cancer can therefore result from the accumulation of a series of specific mutations in the malignant cell.

Oncogenes are genes whose expression causes cells to become cancerous. The normal version of the gene (termed a proto-oncogene) becomes mutated so that it is overactive. Because of their overactivity, oncogenes are genetically dominant over proto-oncogenes, that is only one copy of an oncogene is sufficient to cause a change in the cell's behavior.

The oncogene differs from the normal proto-oncogene in important ways. The coding function of the gene may be unaltered but may be transcribed at a higher rate or under different circumstances from normal. This results in overproduction of a normal gene product. Under other circumstances, there may be under-production of a normal gene product.

Tumor suppressor genes act in a fundamentally different way from oncogenes. Whereas proto-oncogenes are converted to oncogenes by mutations that increase the genes' activity, tumor suppressor genes become oncogenic as the result of mutations that eliminate their normal activity. The normal, unmutated version of a tumor suppressor gene acts to inhibit a normal cell from entering mitosis and cell division. Removal of this negative control allows a cell to divide.

Amplification, overexpression and/or underexpression of some proto-oncogenes and tumor suppressor genes are useful clinically for breast cancer prognosis. However, there is currently no single strong independent marker that is useful for predicting disease outcome in a majority of patients.

The number of clinical laboratory assays currently used in oncology is very small. For breast cancer, only three, namely, estrogen, progesterone and HER2 status are currently assessed. Unfortunately, these three only provide useful prognostic and predictive information in a small number of patients. For example, although HER2 (a proto-oncogene) has emerged as a strong independent prognostic and predictive marker for breast cancer, it is only useful clinically in about 25 to 30% of cases. In the last five years, the College of American Pathologists, the American Society of Clinical Oncology expert panels and the Joint Committee on Cancer have carefully considered many markers proposed for managing breast cancer but have found none with proven clinical utility sufficient to justify their adoption for routine practice and no single marker has been found which gives a consistent result in all manifestations.

There are ongoing studies aimed at identifying new and broad-spectrum markers that will be useful in many cases. But it is unlikely that a single broad-range marker will be found since multiple biochemical pathways are associated with the onset, progression and/or severity of breast cancer. Moreover, the relevant pathways involved may be different in different individuals due to other compounding factors such as aging, race, nutrition, habit and environment.

There is a need in the art for a more sensitive, relatively faster and cost effective approach for generating molecular probes and for assessing the status of breast cancer. There is also a need in the art for an effective approach for assessing the status of multiple prognostic markers in breast cancer.

An object of the present invention is to provide a more sensitive, relatively faster and cost effective approach for assessing the status of prognostic markers in breast cancer. Another object of the present invention is to provide a method and cloning vector for preparing multiple-gene diagnostic probes for the assessment of multiple markers for breast cancer prognosis. These and other objects of the present invention will become more apparent from a consideration of the following description and claims.

### BRIEF SUMMARY OF THE INVENTION

The present invention involves the method and the use of a single cloning vector for preparing multiple-gene diagnostic probes targeting the HER2, Topo IIα, NM23-H1, CK19 and MMP9 genes. The invention permits a cost effective approach for the simultaneous assessment of at least five specific but independent changes in gene profile that enable prognosis. All these changes do not occur together or in sequence in each patient. However, because one detectable change (at least) will occur in greater than 95% of cancers, the invention provides a cost effective approach for a one-step assessment of the molecular status of breast tumors. The present innovation provides a cost effective approach, which is more sensitive and improves the likelihood of detecting molecular changes of prognostic significance in a larger patient population at a relatively lower cost.

The basic elements of the multi-gene probe are the following:

i. labeled fragments complementary to multiple regions of the HER2 gene sequence;

ii. labeled fragments complementary to multiple regions of the Topo IIα gene sequence;

iii. labeled fragments complementary to multiple regions of the NM23-H1 gene sequence;

iv. labeled fragment complementary to the CK19 gene sequence; and

v. labeled fragments complementary to multiple regions of the MMP9 gene sequence;

Each of the gene specific group of fragments is labeled with a different label and each of the fragments is useable to detect its complementary gene sequence. The multi-gene probe may be packaged in the form of an assay kit.

The basic elements of the method of the invention of making the multi-gene probe are the following:

i. sequentially cloning a plurality of DNA fragments complementary to HER2, Topo IIα, NM23-H1, CK19 and MMP9 into a single cloning vector;

ii. transfecting a competent bacterial cell (e.g., an E. coli bacterial cell) with the cloning vector to form a recombinant bacterial cell;

iii. incubating the recombinant bacterial cell in a culture medium to form incubated cells;

iv. isolating the incubated cells from the culture medium;

v. isolating a recombinant vector and generating gene specific oligonucleotide fragments from the cloning vector by PCR or restriction fragment digestion;

vi. separating the linear oligonucleotide fragments by HPLC or gel electrophoresis into individual fragments targeting the HER2, Topo IIα, NM23-H1, CK19 and MMP9 genes; and

vii. labeling each of the individual fragments with a different label, preferably a fluorescent dye, and then mixing them in predetermined concentrations to form the multi-gene probe.

The individual fragments can also be labeled with fluorescence dyes or radioactive nucleotides and used as individual probes for northern or southern analysis.

The method of the invention thus broadly comprises:

(i) cloning DNA fragments unique to HER2, Topo IIαc, NM23-H1, CK19 and MMP9 into a single cloning vector;

(ii) transforming bacteria (E. coli) with the vector and generating large amounts of the vector;

(iii) isolating and separating the oligonucleotide fragments into individual fragments unique to HER2, Topo IIα, NM23-H1, CK19 and MMP9;

(iv) labeling each individual fragment with a different fluorescent label; and

(v) mixing the labeled fragments in predetermined concentrations to form a multi-gene probe.

The basic elements of the cloning vector of the invention for preparing a multi-gene probe for assessing breast cancer prognosis in a human subject are the following:

i. a cloning vector, preferably a plasmid vector; and

ii. target DNA fragments inserted in the vector, preferably at a multiple cloning site of the vector, the DNA fragments comprising a plurality of sequences targeting the HER2, Topo IIα, NM23-H1, CK19 and MMP9 genes.

The multi-gene probe is used to screen a thin section of a tumor specimen, preferably by a fluorescence in situ hybridization (FISH) assay. The resultant signal after imaging has prognostic significance. Since this multi-gene probe targets one or more independent alterations, it provides a one step and highly efficient system for breast cancer prognosis.

The present invention improves and significantly reduces the time and cost associated with the prediction of disease outcome in a larger population of breast cancer patients by enabling detection of multiple changes at the molecular level that correlate with prognosis. The present invention permits a cost effective assessment of at least five independent prognostic markers via a single step.

### BRIEF DESCRIPTION OF THE DRAWINGS

FIG. 1 shows primers for generation of unique oligonucleotide fragments specific for HER2, Topo IIα, NM23-H1, CK19 and MMP9 genes. V5.1, V5.2 and V5.3 are the primers that will be used to generate gene specific fragments for the construction of cloning vectors, versions 1, 2 and 3.

FIG. 2 shows generation, isolation and purification of DNA fragments (of different sizes) unique to HER2, Topo IIα, NM23-H1, CK19 and MMP9 genes. (A) The DNA fragments are generated from genomic DNA by PCR using the PCR primers for V5.1. (B) Each gene specific primer pair is combined with genomic DNA in a separate tube and PCR is carried out. (C) The amplified fragments are purified by gel electrophoresis and restricted with restriction endonucleases to produce cohesive ends.

FIG. 3 shows each DNA fragment restricted with a restriction enzyme to have cohesive and compatible ends.

FIG. 4 shows steps for the insertion of five gene fragments into a cloning vector. All the fragments are inserted by multiple ligation. Each restricted fragment is cloned sequentially (Steps “A” to “E”) into the vector using the same restriction enzyme. For example, (A) HER2 gene specific fragments generated as shown in FIGS. 1 and 2, and restricted with SmaI enzyme, is ligated into a vector that has also been linearized (restricted) with SmaI. In step “B,” the re-circularized vector carrying HER2 is linearized with XbaI to insert TopoIIα. This process is repeated until all fragments are inserted to produce the final product “F.”

FIG. 5 shows the transfection of competent bacteria with the cloning vector. The recombinant vector is mixed with competent E. coli cells and incubated (A) to facilitate transformation of the bacteria (B). The transformed bacteria are incubated in a growth medium and then plated on agar medium (C).

FIG. 6 shows the incubation of the recombinant bacteria in a suitable medium. The transfected and transformed bacteria (A) are transferred to a growth medium, e.g., super broth (B) to generated clones of transformed bacteria each carrying a recombinant vector.

FIG. 7 shows that at the end of incubation (A) the bacteria are pelleted from the growth medium by centrifugation (B) and the recombinant plasmid DNA isolated (C).

FIG. 8 shows the recombinant cloning vector is used as a DNA template for generation of gene specific fragments by PCR. Aliquots of the plasmid DNA are transferred to five tubes and used as the template for PCR. Buffer and the forward and reverse PCR primers specific for each gene are added.

FIG. 9 shows that the PCR amplified fragments are separated from the reaction medium by gel-electrophoresis.

FIG. 10 shows that each generated fragment is labeled with a different fluorescent dye.

FIG. 11 shows a multi-gene probe prepared by mixing predetermined concentrations of each labeled fragment.

### DETAILED DESCRIPTION OF THE INVENTION

The present invention solves the problem of inadequate broad-range prognostic factors for breast cancer by providing a method and strategy for constructing a cloning vector that will be used for preparing multi-gene probes for a single-step determination of disease outcome. The 5-genes chosen are known to show altered expression in different breast tumors. The altered expression of any one of them has a prognostic significance, with respect to disease-free survival or overall survival. The multi-gene probe is labeled and used to screen a breast tumor specimen. The signal generated after the assay serves as a prognostic marker. Since five prognostic markers are simultaneously assessed, this strategy covers a wider variety of breast cancers and so improves disease outcome prediction in a wider population.

The five prognostic markers include HER2. HER2 is an acronym for human epidermal growth factor receptor, also known as c-erbB-2/neu. Growth factors are protein products of genes called proto-oncogenes, which are fundamentally important for normal cells. The proto-oncogenes interact with other genes and their products; these genes, called tumor suppressor genes, also have important roles in normal cell division. HER2 gene amplification and protein overexpression play a pivotal role in oncogenic transformation, tumorigenesis and metastasis. The HER2 gene (ERBB2) maps on chromosome 17q 21.1. The mRNA size is 4.5 kb. The protein expressed by HER2 is a 185-kDa tyrosine kinase receptor for heregulin and other members of the heregulin family.

Topoisomerase IIα (Topo IIα) plays a key role in DNA replication and is a target for multiple chemotherapeutic agents. In breast cancer, Topo IIα expression has been linked to cell proliferation and HER2/neu protein overexpression. Topo IIα (170 kD) maps at chromosome 17q21-q22, and encodes a protein that controls topological states of DNA.

Nm23 is an acronym for nonmetastatic protein 23 or nucleoside diphosphate (NPD) kinase-A (NDPKA). The underexpression of the NM23-H1 gene is related to cell proliferative activity. The NM23-H1 gene maps to 17q22 and consists of 5 exons and 4 introns spanning 8.5 kb. The mRNA size is 0.8 kb. The NM23-H1 gene encodes an about 17 KD protein.

Cytokeratin 19 (CK19) is one of the family of genes for keratins 13, 14, 15, 16, 17, and 19 contained in less than 150 kb of genomic DNA in the region 17q21-q22. The mRNA size is 1.3 kb. The gene expresses a 40-kda acidic keratin component of intermediate filaments. CK19 protein is found on the surface of epithelial cells.

Matrix metalloproteinase-9 (MMP9) is also known as 92-kD gelatinase or Gelatinase B. It is a collagenase type IV-B (CLG4B). In highly metastatic tumor cells, there may be conspicuous expression of MMP9. The MMP9 gene (CLG4B) maps to 20 q11.2-q13.1. MMP9 has 13 exons and similar intron locations. The 13 exons of MMP3 are 3 more than have been found in other member of this gene family. The extra exons encode the amino acids of the fibronectin-like domain, which has been found only in MMP-2 and MMP-9. The mRNA size is 2.8 kb.

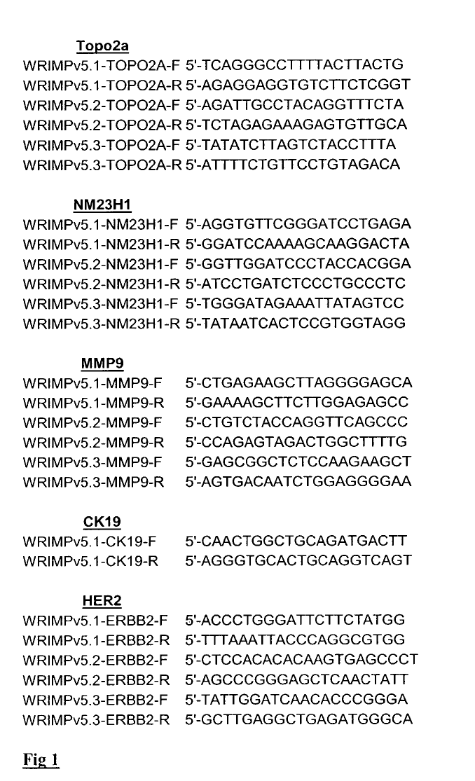

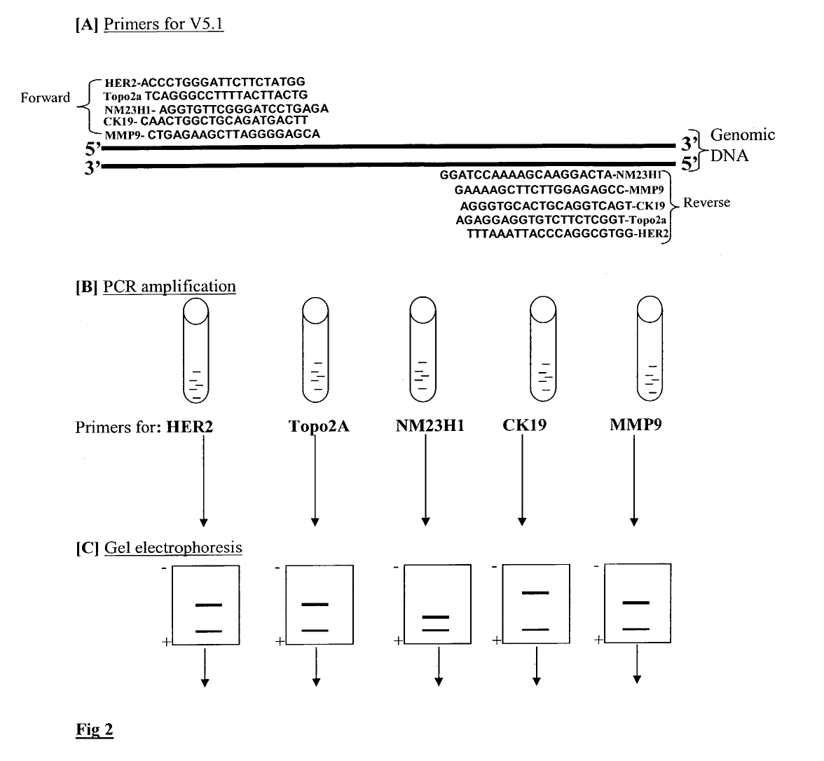

The gene-specific fragments are preferably made by polymerase chain reaction (PCR) using the forward and reverse primers shown in FIGS. 1 and 2. PCR allows an extremely large number of copies to be synthesized of any given DNA sequence. A PCR cycle consists of three steps: (1) denaturation; (2) primer annealing; and (3) elongation. The three steps of the PCR cycle are repeated 25-30 times to obtain large amounts of the target sequence. As more and more reaction cycles are carried out, the target DNA sequence becomes the majority species present. After the PCR reaction is complete, the five fragments are isolated from the reaction mixture and purified by conventional means e.g., by gel electrophoresis.

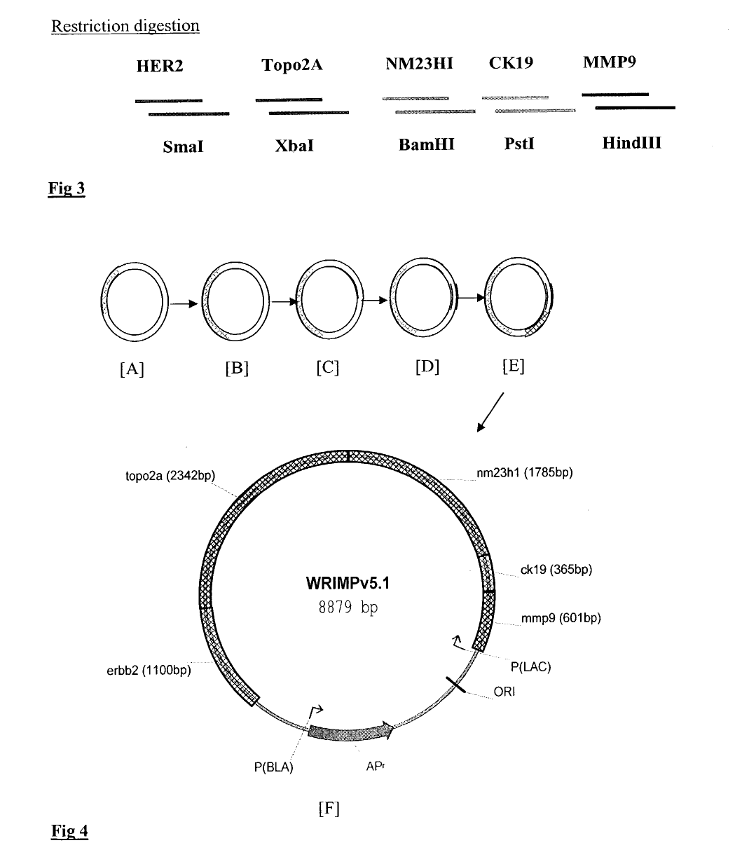

The complementary DNA fragments targeting the breast cancer marker genes are first preferably treated with restriction enzymes (restriction endonucleases) to form compatible cohesive ends as shown in FIG. 3. This type of end can bind to any other end with the same overhanging sequence by base pairing (annealing) of the single-stranded tails. For example, any fragment formed by a restriction enzyme like EcoRI cut can anneal to any other fragment formed by the same enzyme, and may subsequently be joined covalently by ligation. The restriction enzymes preferably are XbaI, BamHI, HindIII, PstI, and SmaI.

DNA ligase enzymes are used for the covalent joining of the gene-specific fragments to the vector at the multiple cloning site. DNA ligase enzymes will join (ligate) one strand of a DNA molecule to another. The five fragments targeting the breast cancer marker genes are thus packaged in a single vector as shown in FIG. 4

Cloning involves placing a relatively short fragment of a genome in an autonomously replicating piece of DNA known as a vector forming recombinant DNA, which can be replicated independently of the original genome, and normally in another host species. Propagation of the host organism containing the recombinant DNA forms a set of genetically identical organisms, or clones.

Any piece of DNA which replicates as a single unit is called a replicon. The initiation of DNA replication within a replicon always occurs at a fixed point known as the origin. Usually, two replication forks proceed bidirectionally away from the origin and the strands are copied as they separate until the terminus is reached. All prokaryotic chromosomes and many bacteriophage and viral DNA molecules are circular and comprise single replicons. Thus, there is a single termination site roughly 180 degrees opposite the unique origin.

A wide variety of natural replicons have the properties required to allow them to act as cloning vectors. Vectors also incorporate a selectable marker, a gene that allows host cells containing the vector to be selected from among those that do not, usually by conferring resistance to a toxin, or enabling their survival under certain growth conditions.

The preferred cloning vector is a naturally occurring bacterial plasmid such as PUC19. Plasmids are small, extrachromosomal circular molecules, from 2 to around 200 kb in size, which exist in multiple copies (up to a few hundred) within the host cell. They contain an origin of replication which enables them to be replicated independently, although this normally relies on polymerases and other components of the host cell's machinery. They usually carry a few genes, one of which may confer resistance to antibacterial substances.

In accordance with the invention, the multiplexed fragment is produced simultaneously by cloning into a suitable cloning vector as shown in FIG. 4. To incorporate fragments of foreign DNA into a plasmid vector, methods for the cutting of DNA are required. This is accomplished by using several restriction endonucleases, which recognizes a short, symmetrical DNA sequence, and cuts (hydrolyzes) the DNA backbone in each strand at a specific site within that sequence. The restriction endonucleases preferably are XbaI, BamHI, HindIII, PstI, and SmaI.

To insert the individual fragments into the vector after cutting, a method for the covalent joining of DNA molecules is essential. DNA ligase enzymes perform this function. These enzymes will repair (ligate) a break in one strand of a DNA molecule. Ligases are efficient at sealing the broken phosphodiester bonds in an annealed pair of cohesive ends, essentially the reverse of a restriction enzyme reaction.

The plasmid vector has a multiple cloning site (MCS). These plasmids have multiple restriction enzymes sites within the first part of the coding region of the gene. Target DNA may be inserted in any of these sites, or between any pair. The use of an MCS allows flexibility in the choice of a restriction enzyme or enzymes for cloning.

The components of the mixture of recombinant and other plasmid molecules formed by ligation must now be isolated from one another and replicated (cloned) by transfer into a host organism. By far the most common hosts are strains of E. coli, which have specific genetic properties. Cells pre-treated with Ca²⁺, in order to render them able to take up DNA by transformation, are known as competent cells.

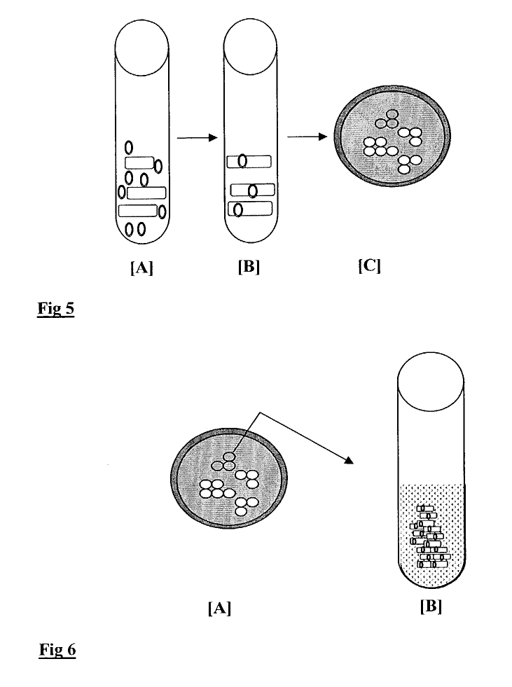

In transformation of E. coli, a solution of a plasmid molecule, or a mixture of molecules formed in a ligation reaction, is combined with a suspension of competent cells for a period to allow the DNA to be taken up. The mixture of transformed bacteria is now plated on an agar plate as shown in FIG. 5. The positively transformed cells will be selected from their distinct color and transferred to a suitable liquid growth medium, e.g., Super broth and incubated to generate large amounts of bacteria as shown in FIG. 6. All the cells within a colony originate from division of a single individual. Thus, all the cells will have the same genotype including the presence of any plasmid introduced in the transformation step (i.e., they will be clones).

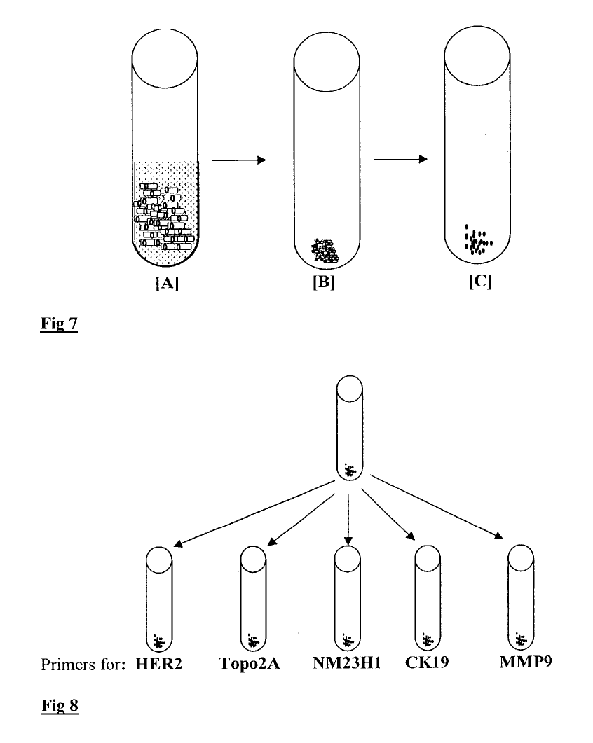

The next step is the isolation of plasmid DNA as shown in FIG. 7. Since plasmids are so much smaller than E. coli chromosomal DNA, they can be separated from the latter by physico-chemical methods, such as alkaline lysis followed by centrifugation. A sample of an E. coli strain harboring the required plasmid is inoculated into a few milliliters of culture broth. After growth to stationary phase (overnight), the suspension is centrifuged to yield a cell pellet, which is purified such as by alkaline lysis to form a supernatant (the lysate). There are many methods for the isolation of pure plasmid DNA from the lysate. The plasmid DNA in the aqueous layer are then concentrated such as by ethanol precipitation.

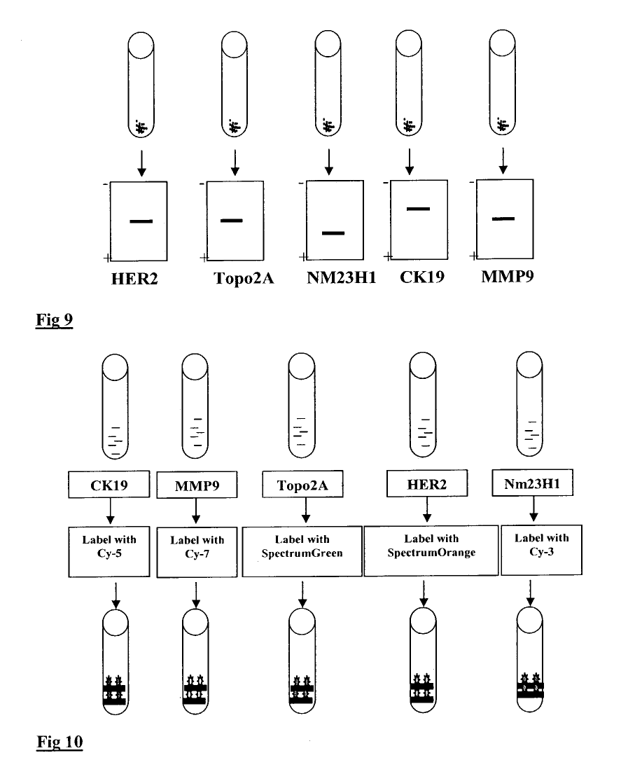

Each cloned fragment is generated by PCR using the gene specific PCR primers as shown in FIG. 8. The amplified PCR fragments can be isolated from the PCR reaction mixture using special PCR clean up spin columns that contain silica gel. DNA binds to the silica gel at acidic pH but can be eluted with water. But the fragments can also be purified by Agarose gel electrophoresis as shown in FIG. 9. Agarose is a polysaccharide derived from seaweed, which forms a solid gel when dissolved in aqueous solution at concentrations between 0.5 and 2% (w/v). Agarose used for electrophoresis is a more purified form of the agar used to make bacterial culture plates. Fragments are excised from the gel, and treated by one of a number of procedures to purify the DNA away from the contaminating agarose and dye mixture.

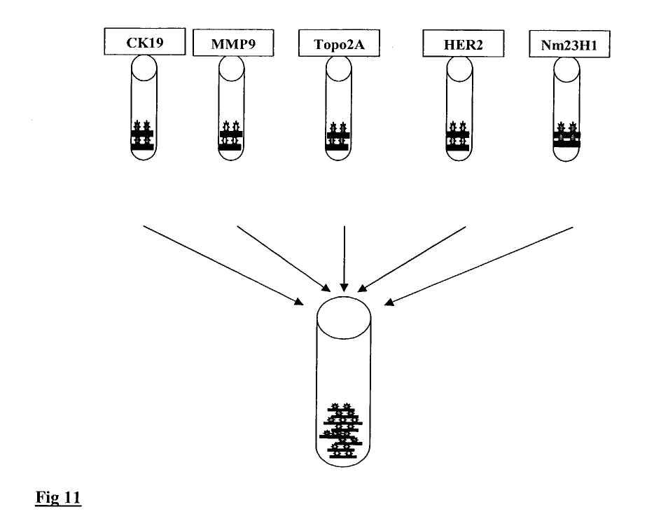

The PCR fragments are recovered from the gel and labeled with different fluorescent dyes as shown in FIG. 10. Preferably, each of the fragments is labeled with a different fluorescent dye as follows:

| | Fluorescent Dye Type | |
|---|---|---|
| Fragment | (Color) | Preferred Fluorescent Dye |
| HER2 | SpectrumOrange ™ | SpectrumOrange ™ (Vysis Inc.) |
| | (Orange) | |
| Topo IIα | SpectrumGreen ™ | SpectrumGreen ™ (Vysis Inc.) |
| | (Green) | |
| NM23-H1 | Cy3 ™ (Red) | Cy3 ™ (Amersham Biosciences) |
| CK19 | Cy5 ™ (Blue) | Cy5 ™ (Amersham Biosciences) |
| MMP9 | Cy7 ™ (Yellow) | Cy7 ™ (Amersham Biosciences) |

A multi-gene diagnostic probe is prepared as illustrated in FIG. 11 by mixing precise concentrations of each labeled fragment as follows:

| | General | Preferred |
|---|---|---|
| Fragment | Concentration Range | Concentration/Experiment |
| HER2 | 10-25 ng | 22 ng |
| Topo IIα | 10-25 ng | 18 ng |
| NM23-H1 | 10-25 ng | 20 ng |
| CK19 | 10-25 ng | 25 ng |
| MMP9 | 10-25 ng | 25 ng |

The multi-gene probe is used to screen a thin section of a tumor specimen. The tumor specimen to be screened is first fixed with, for example, formaldehyde, embedded in wax and then cut into thin sections. The screening is preferably accomplished by in situ hybridization. More specifically, it is possible to incubate radioactive or fluorescent probes with sections of tissues, wash away excess probe and then detect where the probe has hybridized. The most preferred screening technique is by fluorescence in situ hybridization (FISH) assay. The FISH assay broadly comprises denaturation of the specimen DNA, preparation of the probe mixture, hybridization of the specimen DNA and the probe mixture, and post-hybridization washes.

The fluorescent fragments absorb light at a specific excitation wavelength and then emit it at a specific emission wavelength. After unbound probe is removed, the specimen is illuminated at the exciting wavelength to visualize where the fragment has bound. The resultant signal after image analysis has prognostic significance as described in more detail in our copending application cross-referenced above.

The multi-gene probe may be packaged in the form of an assay kit. The kit would typically include the multi-gene probe, a control slide showing HER2 positive cells, a counter stain such as DAPI, NP-40 and a post-hybridization wash buffer such as 20×SSC solution.

While the foregoing invention has been described in some detail for purposes of clarity and understanding, it will be appreciated by one skilled in the art from a reading of this disclosure that various changes in form and detail can be made without departing from the true scope of the invention and appended claims.

We claim:

1. A method of making a multi-gene probe for assessing breast cancer prognosis in a human subject, comprising the steps of:
(a) cloning DNA fragments targeting the HER2, Topo IIα, NM23-H1, CK19 and MMP9 genes into a multiple cloning site of a single cloning vector;
(b) transfecting a competent bacterial cell with the cloning vector to form a recombinant bacterial cell;
(c) incubating the recombinant bacterial cell in a culture medium to form incubated cells;
(d) isolating the incubated cells from the culture medium;
(e) separating linear oligo-nucleotide fragments from the cloning vectors of the isolated incubated cells;
(f) separating the linear oligo-nucleotide fragments into individual fragments targeting the HER2, Topo IIα, NM23-H1, CK19 and MMP9 genes; and
(g) labeling each of the individual fragments with a different fluorescent label and then mixing the labeled fragments in a predetermined concentration to form the multi-gene probe.

2. The method of claim 1 wherein DNA sequences unique to five genes are identified.

3. The method of claim 1 wherein polylinkers are joined to the linear oligo-peptide fragments in step (e) to form a multiplexed fragment.

4. The method of claim 1 wherein the vector is a plasmid.

5. The method of claim 4 wherein the plasmid vector has a multiple cloning site.

6. The method of claim 1 wherein the recombinant bacterial cell is an E.coli cell.

7. The method of claim 1 wherein the individual fragments are purified by high pressure liquid chromatography after separation in step (f).

8. The method of claim 1 wherein the DNA fragments in step (a) complementary to HER2, MMP9, NM23-H1, CK19 and Topo IIα are cloned into a single vector.

9. The method of claim 1 wherein the multi-gene probe is a 5-gene probe.

10. The method of claim 1 wherein each of the individual fragments is labeled with a fluorescent dye.

11. The method of claim 1 wherein the mixed labeled fragments are packaged in an assay kit.

12. The method of claim 1 wherein step (f) is carried out by restriction digestion and step (g) is carried out with restriction endonucleases.

13. A method of making a multi-gene probe for assessing breast cancer prognosis in a human subject, comprising the steps of:
(a) cloning a plurality of fragments targeting the HER2, Topo IIα, NM23-H1, CK19 and MMP9 genes into a multiple cloning site of a PUC19 cloning vector;
(b) transfecting a competent E.coli bacterial cell with the cloning vector to form a recombinant bacterial cell;
(c) incubating the recombinant bacterial cell in a culture medium to form incubated cells;
(d) isolating the incubated cells from the culture medium;
(e) separating linear oligo-nucleotide fragments from the cloning vectors of the isolated incubated cells by restriction digestion;
(f) separating the linear oligo-nucleotide fragments into individual fragments targeting the HER2, Topo IIα, NM23-H1, CK19 and MMP9 genes with restriction endonucleases; and
(g) labeling each of the individual fragments with a different fluorescent label and then mixing the labeled fragments in a predetermined concentration to form the multi-gene probe.

14. A method of making a multi-gene probe for assessing breast cancer prognosis in a human subject, comprising the steps of:
(a) constructing a cloning vector carrying DNA fragments unique to HER2, Topo IIα, NM23-H1, CK19 and MMP9 genes;
(b) cloning the DNA fragments;
(c) separating linear oligo-nucleotide fragments into individual fragments unique to HER2, Topo IIα, NM23-H1, CK19 and MMP9;
(d) labeling each individual fragment with a different label; and
(e) mixing the labeled fragments in a predetermined concentration to form a multi-gene probe.

15. The method of claim 14 wherein the multi-gene probe is a 5-gene probe.

16. The method of claim 14 wherein the DNA fragments are cloned in a plasmid vector.

17. A cloning vector for preparing a multi-gene probe for assessing breast cancer prognosis in a human subject, comprising a vector having target DNA inserted therein, the target DNA comprising a plurality of fragments targeting the HER2, Topo IIα, NM23-H1, CK19 and MMP9 genes.

18. The cloning vector of claim 17 wherein the vector is a plasmid.

19. The cloning vector of claim 17 wherein the fragments are inserted into specific sites in the vector.

20. The cloning vector of claim 17 wherein the fragments are located in unique positions within the multiple cloning site of the vector.

21. A cloning vector for preparing a multi-gene probe for assessing breast cancer prognosis in a human subject, comprising a plasmid vector having target DNA inserted therein at a multiple cloning site, the target DNA comprising unique DNA fragments including:
(a) a fragment complementary to at least a region of the HER2 gene sequence;
(b) a fragment complementary to at least a region of the Topo IIα gene sequence;
(c) a fragment complementary to at least a region of the NM23-H1 gene sequence;
(d) a fragment complementary to at least a region of the CK19 gene sequence; and
(e) a fragment complementary to at least a region of the MMP9 gene sequence.

22. The cloning vector of claim 21 wherein the single vector carries DNA complementary to five genes.
